(19) United States
(12) Patent Application Publication
(10) Pub. No.: US 2026/0207708 A1
(43) Pub. Date: Jul. 23, 2026

(54) THERAPEUTIC USES OF ADIPOSE-SECRETED SIGNALING PROTEIN (ADISSP)

(71) Applicant: UNIVERSITY OF MASSACHUSETTS, Westborough, MA (US)

(72) Inventors: Yong-Xu WANG, Southborough, MA (US); Lei HUANG, Shrewsbury, MA (US)

(21) Appl. No.: 19/143,064

(22) PCT Filed: May 30, 2024
(86) PCT No.: PCT/US2024/031543

§ 371 Date: Jun. 25, 2025

Related U.S. Application Data
(60) Provisional application No. 63/470,671, filed on Jun. 2, 2023.

(51) Int. Cl. A61K 38/19; A61P 1/16; A61P 3/04; A61P 3/10; A61P 9/00

(52) U.S. Cl. CPC A61K 38/19; A61P 1/16; A61P 3/04; A61P 3/10; A61P 9/00

(57) ABSTRACT

Described herein are methods for treating insulin-dependent diabetes in a subject, Donohue syndrome, Rabson-Mendenhall syndrome, or a disorder associated with obesity, preferably diabetes, metabolic syndrome, fatty liver disease, nonalcoholic steatohepatitis (NASH), non-hepatic steatosis, obesity-associated cardiac hypertrophy, or dyslipidemia, the method comprising administering a therapeutically effective amount of adipose-secreted signaling protein (ADISSP) polypeptide to a subject in need thereof. In some embodiments, the polypeptide is administered in a dose of 0.5-2.5 mg/kg/day, e.g., 1.0-1.4 mg/kg per day once a day or once every other day.

### CLAIM OF PRIORITY

This application claims the benefit of U.S. Provisional Patent Application Ser. No. 63/470,671, filed on Jun. 2, 2023. The entire contents of the foregoing are hereby incorporated by reference.

### FEDERALLY SPONSORED RESEARCH OR DEVELOPMENT

This invention was made with Government support under Grant Nos. DK115918 and DK116872 awarded by the National Institutes of Health. The Government has certain rights in the invention.

### TECHNICAL FIELD

Described herein are methods for treating insulin-dependent diabetes in a subject, Donohue syndrome, Rabson-Mendenhall syndrome, or a disorder associated with obesity, preferably diabetes, metabolic syndrome, fatty liver disease, nonalcoholic steatohepatitis (NASH), non-hepatic steatosis, obesity-associated cardiac hypertrophy, or dyslipidemia, the method comprising administering a therapeutically effective amount of adipose-secreted signaling protein (ADISSP) polypeptide to a subject in need thereof. In some embodiments, the polypeptide is administered in a dose of 0.5-2.5 mg/kg/day, e.g., 1.0-1.4 mg/kg per day once a day or once every other day.

### BACKGROUND

People with type 2 diabetes often need more than one medication to manage their glucose levels, and most of them will eventually require insulin. For type 1 diabetes, insulin remains the only established therapy. Moreover, obesity, fatty liver disease, NASH, dyslipidemia, and cardiovascular disease are prevalent in people with diabetes. There is a need for a single drug molecule to concurrently address diabetes and other aspects of metabolic disease.

### SUMMARY

Provided herein are methods of treating a mammalian subject who has insulin-dependent diabetes, Donohue syndrome, or Rabson-Mendenhall syndrome. The methods comprise administering a therapeutically effective amount of adipose-secreted signaling protein (ADISSP) to a subject in need thereof. Also provided is adipose-secreted signaling protein (ADISSP) for use in treating a mammalian subject who has insulin-dependent diabetes, Donohue syndrome, or Rabson-Mendenhall syndrome.

In some embodiments, the subject has type 1 diabetes.

In some embodiments, the subject has a BMI of less than 25, at least 25, or at least 30.

In some embodiments, the subject is human.

In some embodiments, the method comprises administering a polypeptide, optionally comprising a sequence that is at least 80, 85 90, or 95% identical to SEQ ID NO: 2.

In some embodiments, the polypeptide is administered once a day or once every other day.

In some embodiments, the polypeptide is administered parenterally. In some embodiments, the polypeptide is administered intravenously, intramuscularly, or subcutaneously.

In some embodiments, the polypeptide is administered in a dose of 0.5-2.5 mg/kg, e.g., 1.0-1.4 mg/kg, e.g., administered once a day, once every other day, or once every third day.

In some embodiments, the polypeptide comprises one or more modifications.

In some embodiments, the modification comprises one or more of: replacement of one or more L amino acids with D amino acids; acetylation (e.g., comprises an N-acetylalanine at position 2), amidation; conjugation to a linear or branched-chain monomethoxy poly-ethylene glycol (PEG, i.e., PEGylation); modification of the N- or C-terminus; glycosylation: polysialic acid (PSA) addition to a glycan; or fusion to a non-C20orf27 protein, e.g., Fc fusion proteins, fusion to human serum albumin, fusion to transferrin, or fusion to carboxy-terminal peptide of chorionic gonadotropin (CG) β-chain.

Also provided herein are methods of treating a mammalian subject who has a disorder associated with obesity, preferably diabetes, metabolic syndrome, fatty liver disease, nonalcoholic steatohepatitis (NASH), non-hepatic steatosis, obesity-associated cardiac hypertrophy, or dyslipidemia. The methods include administering a therapeutically effective amount of adipose-secreted signaling protein (ADISSP) polypeptide to a subject in need thereof, wherein the polypeptide is administered in a dose of 0.5-2.5 mg/kg, e.g., 1.0-1.4 mg/kg, administered once a day, once every other day, or once every third day. Also provided herein is a composition comprising adipose-secreted signaling protein (ADISSP) polypeptide in a dose of 0.5-2.5 mg/kg, e.g., 1.0-1.4 mg/kg, formulated to be administered once a day, once every other day, or once every third day, e.g., for treating a mammalian subject who has a disorder associated with obesity, preferably diabetes, metabolic syndrome, fatty liver disease, nonalcoholic steatohepatitis (NASH), non-hepatic steatosis, dyslipidemia, or obesity-associated cardiac hypertrophy.

In some embodiments, the subject has a BMI of less than 25, at least 25, or at least 30.

In some embodiments, the subject is human.

In some embodiments, the method comprises administering a polypeptide comprising a sequence that is at least 80, 85 90, or 95% identical to SEQ ID NO:2.

In some embodiments, the polypeptide is administered parenterally. In some embodiments, the polypeptide is administered intravenously, intramuscularly, or subcutaneously.

In some embodiments, the polypeptide comprises one or more modifications. In some embodiments, the modification comprises one or more of: replacement of one or more L amino acids with D amino acids; acetylation (e.g., comprises an N-acetylalanine at position 2), amidation; conjugation to a linear or branched-chain monomethoxy poly-ethylene glycol (PEG, i.e., is PEGylation); modification of the N- or C-terminus; glycosylation; polysialic acid (PSA) addition to a glycan; or fusion to a non-C20orf27 protein, e.g., Fc fusion proteins, fusion to human serum albumin, fusion to transferrin, or fusion to carboxy-terminal peptide of chorionic gonadotropin (CG) β-chain.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a graph showing that ADISSP expression in adipose tissue is negatively correlated with HOMA-IR in human.

FIGS. 2A-B show that Adipose-specific ADISSP knockout (ADKO) mice have elevated glucose level and decreased basal Akt activity. (A) 5-month-old ADKO mice and littermate controls on a normal chow were fasted for 5 hours, and glucose level was measured. (B) Male mice were fasted for 2 hours, Akt phosphorylation was examined. No insulin was injected.

FIGS. 3A-E. ADISSP protein activates Akt independently of insulin receptor, but requires a putative receptor tyrosine kinase (RTK) and PI3K. (A) Akt activation by ADISSP was not blocked by insulin receptor antagonist S961, G protein Gas inhibitor Melittin or Gbg inhibitor Gallein, but was blocked by PI3K inhibitor wortmannin. (B) ADISSP did not activate insulin receptor substrate 1 (IRS1), further suggesting that ADISSP action is independent of insulin and insulin receptor. (C) ADISSP induces a bulk of tyrosine phosphorylation, indicating that ADISSP might activate a receptor tyrosine kinase. (D) A pan-inhibitor of receptor tyrosine kinase LDC1267 blocks ADISSP-induced Akt activation, indicating that ADISSP activates a receptor tyrosine kinase. Serum-starved brown adipocytes were treated with ADISSP protein (300 nM) or insulin (100 nM) for 30 min. In (A) and (D), adipocytes were pre-treated with inhibitors for 2 hr. S961 (0.5 μM), Melittin (1 μM), Gallein (5 μM), wortmannin (1 μM), and LDC1267 (1 μM). (E) A simplified model of ADISSP action. ADISSP uses a downstream mechanism similar as the one used by insulin, but independently of insulin receptor and insulin receptor substrates, to lower blood glucose.

FIG. 4. Purified ADISSP protein induces Glut4 trafficking to the plasma membrane.

FIGS. 5A-C. A single dose (70 nmole/kg) of recombinant ADISSP protein induces Akt activation in adipose tissue without stimulating insulin secretion. (A) ADISSP protein lowers blood glucose level in euglycemic mice without causing hypoglycemia. (B) ADISSP protein activated Akt in vivo. BAT, brown fat; iWAT, subcutaneous inguinal white fat; eWAT, epididymal white fat. (C) ADISSP protein did not stimulate insulin secretion, dissociating it from the incretins on the market.

FIGS. 6A-D. Injection of purified ADISSP protein normalizes hyperglycemia, improves hyperinsulinemia, and reduces liver steatosis in diabetic ob/ob mice. N=15 mice/group. (A) The glucose-lowering effect of a single dose (50 nmole/kg) of ADISSP was maintained for more than 20 hours. (B) 46 hours post the first injection, ob/ob mice were injected with ADISSP (50 nmole/kg per injection) once every 48 hour at 8 am (arrow). Mice were fed ad libitum. Glucose was lowered by 83.5 mg/dl on average. After each subsequent injection, the glucose-lowering effect was maintained for at least 48 hours. (C) Liver steatosis was also improved, as all of the livers in the treatment group were darker, indicating reduced lipid accumulation, and liver histology showed reduced lipid accumulation. (D) Improvement in hyperinsulinemia in mice treated with ADISSP as compared to vehicle.

FIGS. 7A-G. One-month treatment of hyperglycemic ob/ob mice with ADISSP protein. Vehicle or ADISSP protein (62.6 nmole/kg per day) was intraperitoneally injected once a day at 8 am for 30 days; no daily insulin was administered. Glucose levels were measured at 7 am daily. N=14-16 mice/group. (A) Daily glucose levels. (B) Glucose tolerance test (58.4 mg glucose per mouse) done at the end of the experiments, showing that ADISSP treatment greatly improves glucose tolerance. (C) Insulin tolerance test (0.75 U/kg insulin per mouse) done at the end of the experiments, showing that ADISSP treatment greatly improves insulin sensitivity as well. Data were presented as percentage. (D) Daily food intake. (E) Body weight gain. Initial body weights were 52.51±0.81 g for the control group, and 54.69±1.11 g for the treatment group. (F) Liver histology and weight show that ADISSP treatment reverses fatty liver. (G) No loss of bone density was seen after 30 days of treatment with ADISSP.

FIGS. 8A-C. Two-week treatment of high fat-induced obese (DIO) mice with purified ADISSP protein. Mice were intraperitoneally injected with ADISSP protein (65 nmole/kg per day) once a day at 8 am for 14 days. N=19 DIO mice/group. (A) daily glucose levels and (B) improvement of hyperinsulinemia, and (C) amelioration of fatty liver were all evaluated.

FIGS. 9A-K. One-month treatment of hyperglycemic DIO mice with purified ADISSP protein. Glucose levels were measured every day at 7:30 am followed by intraperitoneal injection of ADISSP (71 nmole/kg per day). N=19-20 DIO mice. (A) daily glucose levels. (B) Daily food intake (C) Glucose tolerance test. (D) Insulin tolerance test. (E) Body weight gain. (F) Fat mass and lean mass, showing that ADISSP treatment reduces fat mass, but not lean mass. (G) Body surface temperature showed that energy was dissipated as heat. (H) Core body temperature showed no fever. (I) Amelioration of fatty liver. (J) Reduced serum triglycerides. (K) Reduced liver damage.

FIGS. 10A-B. ADISSP protein markedly lowers blood glucose in streptozotocin (STZ)-induced type 1 diabetic mice. (A) glucose levels in mice administered a single dose (200 mg/kg) of STZ. Mice were then intraperitoneally injected with ADISSP protein (50 nmole/kg per injection) as shown. (B) glucose levels in mice administered a low dose (50 mg/kg) of STZ injected daily from Day 1 to Day 5. ADISSP protein (70 nmole/kg per day) was injected daily from Day 13. In both of these two experiments, polyuria caused by diabetes was greatly improved.

FIGS. 11A-E. Adissp protein treatment resolves NASH and cardiac hypertrophy. (A) Blood glucose. (B) Liver size and weight. (C) Liver histology and steatosis and inflammation scores. (D) Sirius red staining and quantification. (E) Heart weight and size.

### DETAILED DESCRIPTION

Insulin therapy is associated with glucose variability, hypoglycemia, and body weight gain. Currently only about 50% of people with type 2 diabetes and about 20% of people with type 1 diabetes achieve optimal glycemic outcomes. Therefore, there is a need to develop new drugs, in particular drugs that use a novel mechanism of action, to complement existing diabetic drugs and provide more therapeutic options. Conceivably, a molecule with insulin-like activity but acting independently of the insulin receptor has the potential to allow for lower insulin requirements and offer better glycemic control without risking hypoglycemia and body weight gain. Ensuing lower insulin usage could also potentially prevent insulin resistance and delay disease progression.

In addition, people with type 2 diabetes have a 80-90% prevalence of overweight/obesity and a 60-68% prevalence of nonalcoholic fatty liver disease (NAFLD). People with type 1 diabetes also have a rising prevalence of overweight/obesity and NAFLD, which further accelerate the development of type 1 diabetes and increase the risks of cardiovascular disease and microvascular complications. A diabetic drug that can concurrently address obesity and NAFLD would be ideal.

As shown herein, adipose-secreted signaling protein (ADISSP, formerly c20of27 in humans) robustly activates Akt independently of insulin and insulin receptor and has a long-lasting and sustainable glucose-lowering effect in diabetic mouse models, while concurrently reducing body weight and ameliorating fatty liver. Unlike insulin, it does not cause hypoglycemia or body weight gain, nor does it stimulate insulin secretin like incretins. Thus, administration of ADISSP can allow for lower insulin requirements and offer better glycemic control without risking hypoglycemia and body weight gain, and the ensuing lower insulin usage could also potentially prevent insulin resistance and delay disease progression.

As shown herein, ADISSP protein activates Akt and its downstream targets and induces Glut4 plasma membrane localization independently of insulin and insulin receptor, a novel mechanism of action that was not used by current diabetes therapeutics. Without wishing to be bound by theory, it is believed that ADISSP protein activates two beneficial pathways in adipose tissue: thermogenesis pathway to promote energy expenditure and Akt pathway to lower glucose.

ADISSP protein begins to lower glucose level two hours post injection, and the effect of a single injection can last for more than 20 hours. Injection of ADISSP protein once every 48 hours was sufficient to normalize hyperglycemia. The glucose lowering effect is long-lasting and sustainable, and is independent of food intake. The dosage of ADISSP (1.0-1.4 mg/kg per day, that is, 50-70 nmole/kg per day) used in the present study was similar to many published studies that treat diabetic mice with biologics (e.g., FGF21).

Administration of ADISSP protein does not stimulate insulin secretion, dissociating it from the incretins on the market. In addition, ADISSP does not cause hypoglycemia, but improves insulin sensitivity, and thus can be used to lower insulin requirements. The ensuing lower insulin usage could bypass the side effects associated with insulin therapy, prevent insulin resistance, and delay disease progression.

In addition, ADISSP treatment reverses fatty liver and liver damage and normalizes hypertriglyceridemia. Unlike FGF21 or commonly-prescribed thiazolidinedione diabetes therapeutics, ADISSP has no side effects on bone density as assayed by X-ray.

Based on these results, ADISSP is a therapeutic biologic for the treatment of both type 1 and type 2 diabetes, NAFLD, NASH, dyslipidemia and obesity, e.g., using a once a day or once-every other day administration regimen. In addition, Donohue syndrome and Rabson-Mendenhall syndrome are rare diseases characterized by severe insulin resistance and hyperglycemia, due to mutations in the insulin receptor gene. Currently, there are no effective treatments for either. The mechanism of action and glucose lowering effect demonstrated herein make ADISSP a therapeutic option for treatment of Donohue syndrome and Rabson-Mendenhall syndrome.

### Adipose-Secreted Signaling Protein (ADISSP)

ADISSP is exclusively expressed in the adipose tissue with a significant and high enrichment in brown fat versus white fat. The polypeptide is encoded by an open reading frame in humans that encodes two isoforms. Isoform 2 is a 19 Kd protein with 174 amino acids, and shares 90% identity between mouse and human. Exemplary sequences of human ADISSP are shown in Table 1.

TABLE 1
Exemplary human ADISSP sequences

| Variant | NCBI RefSeq ID | Isoform | NCBI RefSeq ID |
|---|---|---|---|
| 1 | NM_001039140.2 | 1 | NP_001034229.1 |
| 2 | NM_001258429.1 | 2 | NP_001245358.1 |
| 3 | NM_001258430.1 | 2 | NP_001245359.1 |

Variant 1 encodes the longer isoform 1. Variant 2 uses an alternate splice site in the coding region, and variant 3 differs in the 5′ UTR and uses an alternate splice site in the coding region, but variants 2 and 3 maintain the same reading frame as variant 1. Variants 2 and 3 encode the same isoform 2, which is shorter than isoform 1. Exemplary protein sequences for isoforms 1 and 2 are shown below. As shown in uppercase letters, isoform 1 has a 25-amino acid extension at the N-terminus compared with isoform 2.

```
NP_001034229.1 -- UPF0687 protein C20orf27
(ADISSP) isoform 1
(SEQ ID NO: 1)
    1 MAAANKGKCL PGVVGLAQAL PVGPGrraia
        agnkprvrei rfaaghdaeg shshvhfdex
  61 lhdsvvmvtq esdssflvkv gflkilhrye
        itftlppvhr lskdvreapv pslhlkllsv
121 vpvpegysvk ceysahkegy lkeeillace
        ggtgtcvrvt vqarvmdrhh ctpmlldgvk
131 cvgaeleyds ehsdwhgfd
NP_001245358.1 -- UPF0687 protein C20orf27
(ADISSP) isoform 2
(SEQ ID NO: 2)
    1 maaankgnkp rvrsirfaag hdaegshshv
        hfdeklhdsv vmvtqesdss flvkvgflki
  61 lhryeitftl ppvhrlskdv reapvpslhl
        llsvvpvpe gysvkceysa kkegvlkeei
121 llaceggtgt cvrvtvqarv mdrhhgtpml
        ldgvkcvgae leydsehsdw hgfd
```

In some embodiments, isoform 2 or an active fragment thereof is used. In some embodiments, isoform 1 or an active fragment thereof is used. Active fragments are those that induce the expression of Ucp1 in cultured white adipose cells. In some embodiments, the active fragment includes the “Domain of unknown function (DUF4517); pfam15006,” i.e., amino acids 28-173 of isoform 2, or amino acids 53-198 of isoform 1. In some embodiments, isoform 2 is used.

Additional homologs of ADISSP are provided in Table 2.

TABLE 2
Homologs of Human ADISSP

| Homolog, species | GenBank Acc. No. |
|---|---|
| Full name | Length in aa |
| C20orf27, H. sapiens | NP_001034229.1 |
| chromosome 20 open reading frame 27 | 199 aa |
| C20H20orf27, P. troglodytes | XP_530265.2 |
| chromosome 20 open reading frame, human | 199 aa |
| C20orf27 | |
| C10H20orf27, M. mulatta | XP_001115327.1 |
| UPF0687 protein C20orf27-like | 199 aa |
| C24H20orf27, C. lupus | XP_005634887.1 |
| chromosome 24 open reading frame, human | 190 aa |
| C20orf27 | |
| C13H20orf27, B. taurus | NP_001039670.1 |
| chromosome 13 open reading frame, human | 174 aa |
| C20orf27 | |
| 1700037H04Rik, M. musculus | NP_080367.1 |
| RIKEN CDNA 1700037H04 gene | 174 aa |
| RGD1311739, R. norvegicus | NP_001020862.1 |
| similar to RIKEN cDNA 1700037H04 | 174 aa |
| LOC100858801, G. gallus | XP_003643512.2 |
| UPF0687 protein C20orf27 homolog | 380 aa |
| c20orf27, X. tropicalis | NP_001007504.1 |
| chromosome 20 open reading frame 27 | 174 aa |
| LOC560941, D. rerio | XP_689433.5 |
| UPF0687 protein C20orf27 homolog | 173 aa |

The ADISSP compositions used in the methods described herein can include a peptide that is at least 80%, e.g., at least 85%, 90%, or 95% identical to the amino acid sequence of SEQ ID NO: 1 or SEQ ID NO:2, e.g., have differences at up to 5%, 10%, 15%, or 20% of the residues of SEQ ID NO:1 or SEQ ID NO:2 replaced, e.g., with conservative mutations, or deleted. Alternatively, the compositions can include nucleic acids that encode peptide that is at least 80%, e.g., at least 85%, 90%, or 95% identical to the amino acid sequence of SEQ ID NO: 1 or SEQ ID NO:2, e.g., have differences at up to 5%, 10%, 15%, or 20% of the residues of SEQ ID NO:1 or SEQ ID NO: 2 replaced, e.g., with conservative mutations, or deleted. The variants useful in the present methods retain a desired activity of the parent, e.g., the ability to induce Glut4 plasma membrane localization.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul et al. (1990) J Mol Biol 215:403-10), BLAST-2, BLAST-P, BLAST-N, BLAST-X, WU-BLAST-2, ALIGN, ALIGN-2, CLUSTAL, or Megalign (DNASTAR) software. See, e.g., Altschul et al. (2005) FEBS J. 272:5101-5109. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned.

For purposes of the present invention, the comparison of sequences and determination of percent identity between two sequences can be accomplished using a Blosum62 scoring matrix with a gap penalty of 11,1.

In some embodiments, the protein includes one or more modifications, e.g., is acetylated (e.g., comprises an N-acetylalanine at position 2), amidated, conjugation to either linear or branched-chain monomethoxy poly-ethylene glycol (PEG, i.e., PEGylation), modification of the N- or C-terminus, glycosylation, polysialic acid (PSA) addition to a glycan, or fusion proteins, e.g., Fc fusion proteins, fusion to human serum albumin, fusion to carboxy-terminal peptide, and other polypeptide fusion approaches to make drugs with more desirable pharmacokinetic profiles; see, e.g., Werle and Bernkop-Schnürch, Amino Acids. 2006 June; 30 (4): 351-67; Strohl, BioDrugs. 2015; 29 (4): 215-239; Wang et al., Signal Transduction and Targeted Therapy 7:48 (2022).

### Methods of Treatment

The methods described herein include methods for the treatment of diabetes, e.g., insulin-dependent diabetes, and other disorders. In some embodiments, the disorder is type 1 diabetes, e.g., autoimmune diabetes, e.g., diabetes not associated with obesity or metabolic syndrome. In some embodiments, the disorders are Donohue syndrome or Rabson-Mendenhall syndrome. In some embodiments, the disorder is a disorder associated with obesity, preferably type 2 (or non-insulin dependent) diabetes, metabolic syndrome, fatty liver disease, nonalcoholic steatohepatitis (NASH), non-hepatic steatosis, dyslipidemia, or obesity-associated cardiac hypertrophy.

Generally, the methods include administering a therapeutically effective amount of an ADISSP peptide or nucleic acid encoding the ADISSP peptide as described herein, to a subject who is in need of, or who has been determined to be in need of, such treatment. In some embodiments, the methods include administering a therapeutically effective amount of adipose-secreted signaling protein (ADISSP) polypeptide, e.g., in a dose of 0.5-2.5 mg/kg/day, e.g., 1.0-1.4 mg/kg per day once a day or once every other day.

As used in this context, to “treat” means to ameliorate at least one symptom of diabetes, e.g., hyperglycemia; thus, a treatment can result in a reduction in blood glucose levels and a return or approach to normoglycemia.

In some embodiments, the subjects treated by the methods described herein have insulin-dependent diabetes mellitus (IDDM), i.e., produce no endogenous insulin. In some embodiments, the subjects treated by the methods described herein are being treated for IDDM, e.g., have been prescribed or are taking insulin. In some embodiments, the methods include administering no insulin or a reduced dose of insulin, e.g., 10%, 20%, 25%, 50%, 75%, or 80% of the dose required in the absence of administration of ADISSP.

In some embodiments, the subjects treated by the methods described herein have or are being treated for obesity, metabolic syndrome, or non-insulin dependent or type 2 diabetes, e.g., have been prescribed or are taking a diabetes therapeutic, e.g., semaglutide, tirzepatide, meglitinides, biguanides, thiazolidinediones, sodium-glucose co-transporter-2 inhibitors, or alpha-glucosidase inhibitors. In some embodiments such subjects have also been prescribed insulin. In some embodiments, the methods include administering no insulin or a reduced dose of insulin, e.g., 10%, 20%, 25%, 50%, 75%, or 80% of the dose required in the absence of administration of ADISSP, or no or a reduced dose of another diabetes therapeutic. A person who is diabetic has one or more of a Fasting Plasma Glucose Test result of 126 mg/dL or more; a 2-Hour Plasma Glucose Result in an Oral Glucose Tolerance Test of 200 mg/dL or more; and blood glucose level of 200 mg/dL or above.

In some embodiments the subjects are pre-diabetic, e.g., they have impaired glucose tolerance or impaired fasting glucose, e.g., as determined by standard clinical methods such as the intravenous glucose tolerance test (IVGTT) or oral glucose tolerance test (OGTT), e.g., a value of 7.8-11.0 mmol/L two hours after a 75 g glucose drink for impaired glucose tolerance, or a fasting glucose level (e.g., before breakfast) of 6.1-6.9 mmol/L. Subjects who have or are at risk for insulin resistance or impaired glucose tolerance are readily identifiable, and the treatment goals are well defined.

In some embodiments the subjects have nonalcoholic fatty liver disease (NAFLD) and its most severe form, nonalcoholic steatohepatitis (NASH).

In some embodiments the subjects are not pre-diabetic, do not have metabolic syndrome; do not have fatty liver disease; and/or are not obese. BMI is determined by weight relative to height, and equals a person's weight in kilograms divided by height in meters squared (BMI=kg/m²); in some embodiments, the subjects treated herein have a BMI of below 30, or below 25. Accepted interpretations are given in Table 3.

TABLE 3

| Category | BMI |
|---|---|
| Underweight | ≤18.5 |
| Normal weight | 18.5-24.9 |
| Overweight | 25-29.9 |
| Obese | ≥30 |

As shown herein, ADISSP can improve glucose homeostasis in both insulin-dependent and non-insulin dependent diabetic subjects, thus ADISSP can be used to improve glycemic control in type 1 diabetic patients (even without insulin or with a greatly reduced dose of insulin), e.g., using only once-a-day or once every other day dose administration as described herein. This includes improving the maintenance of blood glucose levels within a desired range, e.g., maintaining a hemoglobin A1c (HbA1c) level below a desired range, e.g., below 7%.

### Gene Therapy

The nucleic acids described herein, e.g., nucleic acids encoding an ADISSP polypeptide or active fragment thereof, can be incorporated into a gene construct to be used as a part of a gene therapy protocol. The present methods can include the use of expression vectors for in vivo gene delivery and expression of a polynucleotide that encodes an ADISSP polypeptide or active fragment thereof, as described herein, e.g., in particular cell types, especially hepatic or adipose cells, as described in WO 2018/191599.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, the dose is 0.5-2.5 mg/kg, or 1.0-1.4 mg/kg per day, that is, 25-125 nmole/kg or 50-70 nmole/kg, e.g., per day or per every two days. In some embodiments, the dose is administered once a day, or divided and administered twice a day, or administered once every other day, e.g., about every 48 hours, in a dose of 0.5-2.5 mg/kg.

### Pharmaceutical Compositions and Methods of Administration

The methods described herein include the manufacture and use of pharmaceutical compositions, which include ADISSP peptides described herein as active ingredients. Also included are the pharmaceutical compositions themselves.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of preferred routes of administration include parenteral, e.g., intravenous, intramuscular, or subcutaneous administration. Methods of formulating suitable pharmaceutical compositions are known in the art, see, e.g., the books in the series Drugs and the Pharmaceutical Sciences: a

Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In some embodiments, the ADISSP peptides are formulated with, e.g., liposomes or micelles. Biodegradable microparticle or nanoparticle delivery systems that increase intracellular uptake, e.g., polymeric and surface modified nanoparticles as described in US 2009/0136585, can also be used. Examples include poly DL-lactide-co-glycolide (PLGA) nanoparticles, e.g., surface-modified with known surface-modifying agents, such as heparin, dodecylmethylammonium bromide (DMAB), DEAE-Dextran, lipofectin, and fibrinogen (see, e.g. Song et al., J. Control. Release, 54:201-211 (1998); Labhasetwar et al., J. Pharm. Sci., 87:1229-34 (1998); Lee et al., Biomaterials 29 (9): 1224-1232 (2008); and US 2009/0136585.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration. Also provided herein are single unit dose forms comprising a dose of 0.5-2.5 mg/kg, or 1.0-1.4 mg/kg.

### EXAMPLES

The invention is further described in the following examples, which do not limit the scope of the invention described in the claims.

### Example 1. Therapeutic Potential of Recombinant ADISSP Protein for the Treatment of Type 1 and Type 2 Diabetes

ADISSP (adipose-secreted signaling protein, previously known as c20orf27), is almost exclusively expressed in mouse adipose tissue and is highly enriched in brown fat in mice; in humans, ADISSP is selectively expressed in adipose tissue. ADISSP is effectively secreted from mouse and human adipocytes and is present in human serum.

ADISSP was shown to promote thermogenic and glycolytic gene expression in adipose tissue (Chen, Q. et al., Nature Communications 13:7633 (2022)), however its actions outside of thermogenesis were not fully explored.

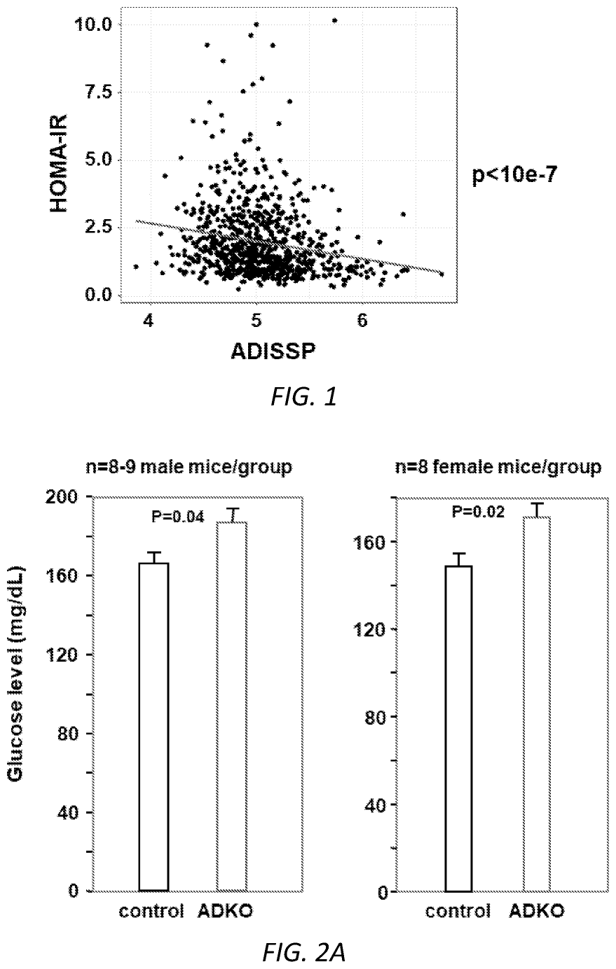

First, linear regression analysis was performed on a public dataset (Civelek, M. et al. Am J Hum Genet 100, 428-443 (2017)); the results, shown in FIG. 1, demonstrated that ADISSP expression levels in subcutaneous adipose tissue of 770 men negatively correlated with HOMA-IR (Homeostatic Model Assessment for Insulin Resistance).

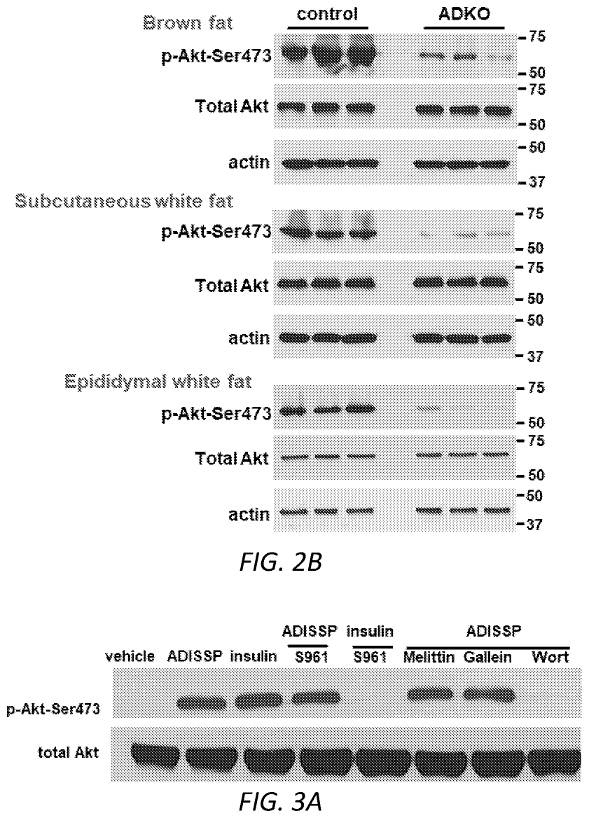

Glucose levels and basal Akt activity were evaluated in adipose-specific ADISSP knockout (ADKO) mice (see WO 2018/191599 and Chen et al., 2022). 5-month-old ADKO mice and littermate controls on a normal chow were fasted for 5 hours, and glucose levels were measured. As shown in FIG. 2A, the ADKO mice were elevated blood glucose level. In addition, Male mice were fasted for 2 hours, and then Akt phosphorylation, which reflects Akt activity, was examined. No insulin was injected. The ADKO mice had decreased basal Akt activity in adipose tissue as shown in FIG. 2B, indicating that ADISSP plays a direct role in regulating Akt activity and glucose homeostasis independently of thermogenesis, as thermogenesis of ADKO mice was not altered at room temperature.

To study the pharmacological effects of ADISSP protein, we purified recombinant ADISSP protein from medium of transiently transfected Expi293F™ (Gibco) cells cultured in suspension. Briefly, a Histidine (His)-tag was added to the C-terminus of ADISSP cDNA, and endotoxin-free plasmids were purified. Plasmids were transfected into Expi293F™ cells, and the cells were cultured in Expi293 expression medium (Gibco), which is serum-free. ADISSP protein was purified from the medium with Ni-NTA beads at 4° C., and eluted with buffer (10% Glycerol, 20 mM Tris pH 7.5, 250 mM Imidazole, and 500 mM NaCl). Eluted ADISSP protein was buffer-exchanged to buffer containing 20 mM HEPES pH 7.5, and 150 mM NaCl at 4° C. with final residue Imidazole level calculated at 0.06 μM.

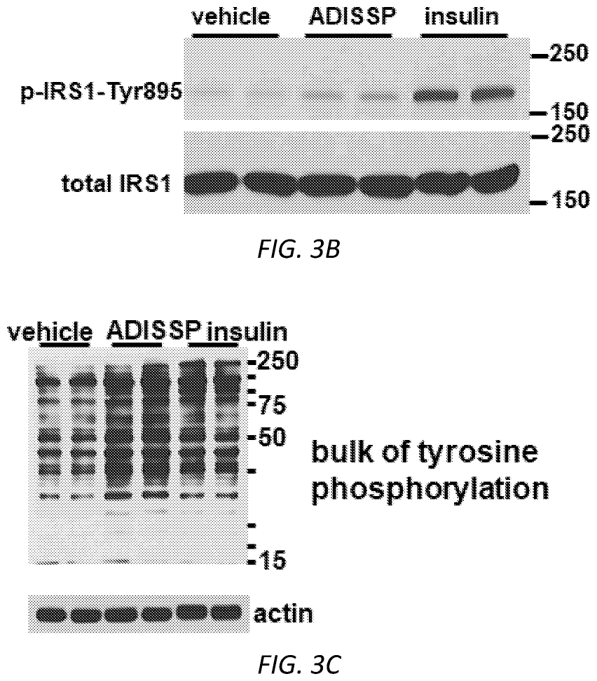

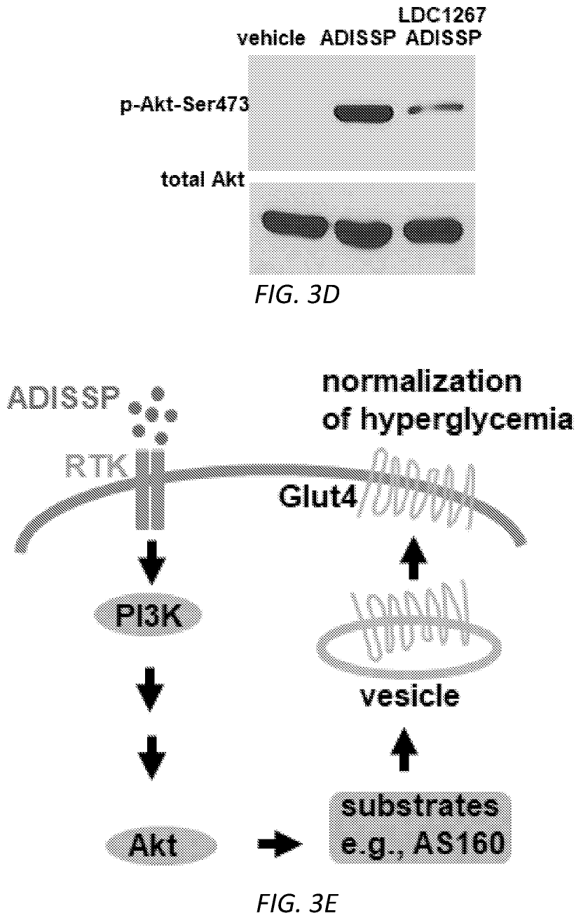

To determine whether ADISSP protein activates Akt independently of insulin receptor, serum-starved brown adipocytes were treated with purified ADISSP protein (300 nM) or insulin (100 nM) for 30 min. In some cases adipocytes were pre-treated with inhibitors for 2 hr: S961 (0.5 μM), Melittin (1 μM), Gallein (5 μM), wortmannin (1 μM), and LDC1267 (1 μM). As shown in FIG. 3A, Akt activation by ADISSP was not blocked by insulin receptor antagonist S961, G protein Gas inhibitor Melittin or Gbg inhibitor Gallein, but was blocked by PI3K inhibitor wortmannin. As shown in FIG. 3B, ADISSP did not activate insulin receptor substrate 1 (IRS1), further suggesting that ADISSP action is independent of insulin and insulin receptor. As shown in FIG. 3C, ADISSP induces a bulk of tyrosine phosphorylation, indicating that ADISSP might activate a receptor tyrosine kinase. As shown in FIG. 3D, a pan-inhibitor of receptor tyrosine kinase LDC1267 blocks ADISSP-induced Akt activation, indicating that ADISSP activates a receptor tyrosine kinase. These results show that ADISSP activates a putative receptor tyrosine kinase (RTK)-PI3K-Akt signaling cascade, analogous to insulin-insulin receptor-IRS-PI3K-Akt pathway.

Without wishing to be bound by theory, FIG. 3E provides a simplified model of ADISSP action. ADISSP uses a downstream mechanism similar as the one used by insulin, but independently of insulin receptor and insulin receptor substrates, to lower blood glucose.

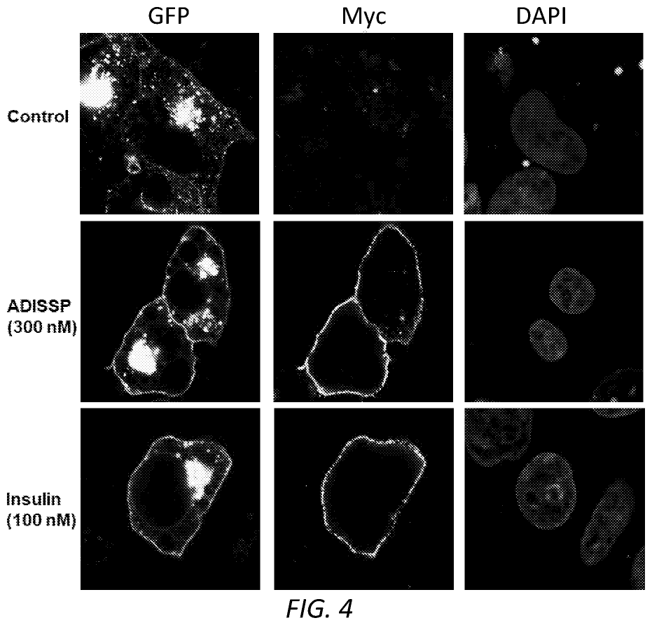

To evaluate effects of ADISSP on Glut4 trafficking to the plasma membrane, adipocytes were transfected with a Myc-Glut4-GFP plasmid, in which Myc and GFP were fused to the extracellular and intracellular regions of Glut4, respectively. The Adipocytes were serum-starved overnight, and then treated with purified ADISSP protein or insulin for 30 min. The adipocytes were fixed without permeabilization, and stained with Myc antibody and DAPI. The GFP signal indicates transfected cells and total Glut4 signal, and Myc signal indicates Glut4 localization; as shown in FIG. 4, both ADISSP and insulin induced Glut4 localization to the plasma membrane. 95% of GFP-positive cells were also Myc-positive after either ADISSP or insulin treatment, while very few GFP-positive cells were Myc-positive in control samples. These results demonstrate that ADISSP protein, through activating Akt, induces Glut4 trafficking to the plasma membrane for glucose uptake, analogous to the effect of insulin, despite that they utilize different receptors.

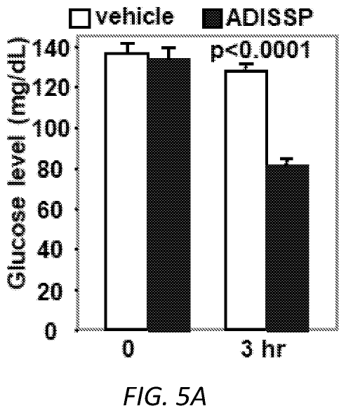

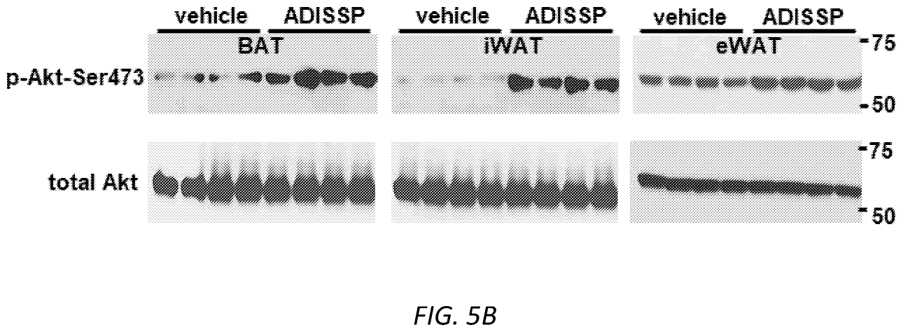

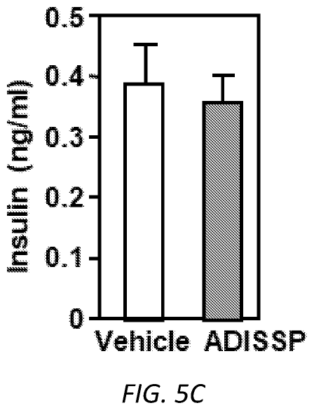

To explore the mechanism of ADISSP action in vivo, 3-month-old male mice (n=4/group) with euglycemia were intraperitoneally (ip) injected with purified ADISSP protein (70 nmole/kg) at 0 hr. Glucose and insulin levels and Akt activation were measured 3 hr after injection. As shown in FIG. 5A, ADISSP protein lowers blood glucose level in euglycemic mice without causing hypoglycemia. FIG. 5B shows that ADISSP protein activated Akt in vivo. However, FIG. 5C shows that ADISSP protein did not stimulate insulin secretion, dissociating it from the incretins on the market. Thus, ADISSP protein activates Akt and lowers blood glucose level but does not cause hypoglycemia.

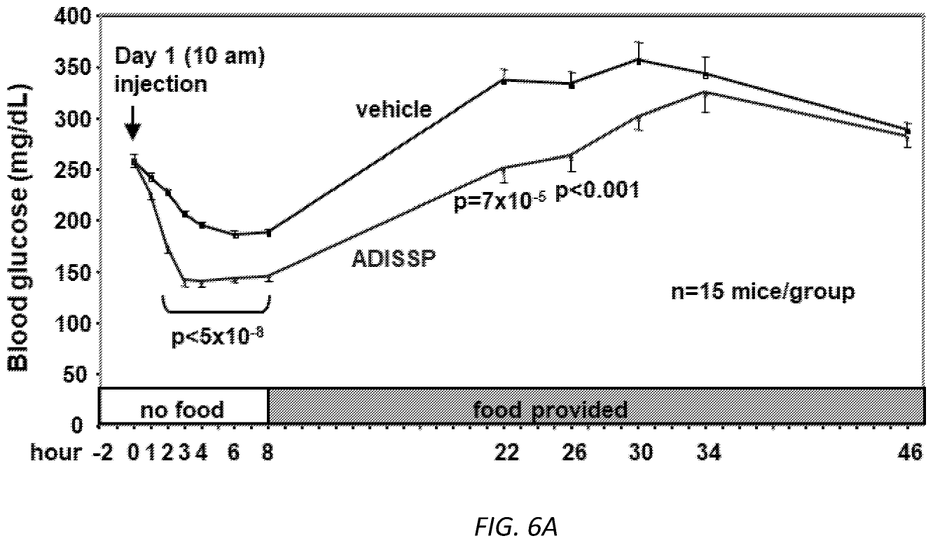

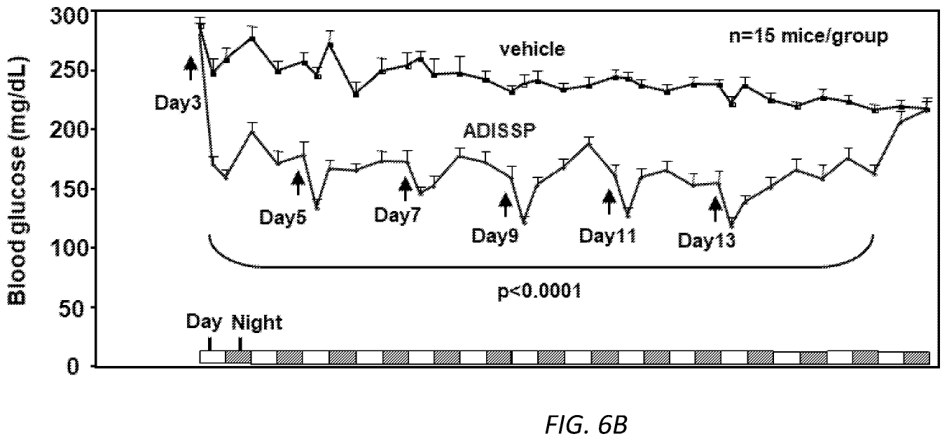

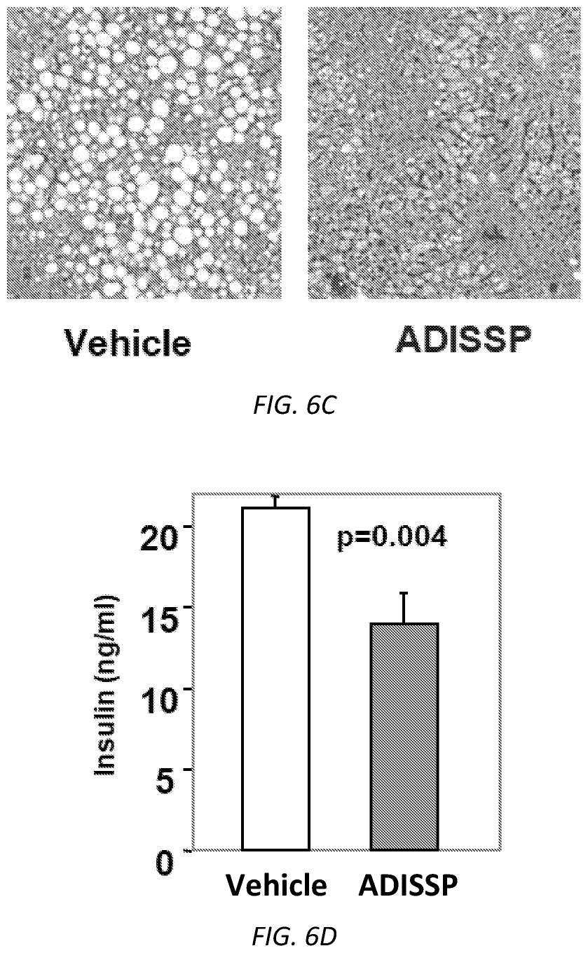

The effects of injection of purified ADISSP protein were evaluated on hyperglycemia, hyperinsulinemia, and liver steatosis in ob/ob mice (6 male and 9 female mice in control group and 5 male and 10 female mice in treatment group), which were used as a type 2 diabetic mouse model. The mice were injected intraperitoneally with vehicle or a single dose of purified ADISSP protein (50 nmole/kg). Glucose was monitored for 46 hours. Note, no food was provided in the first 8 hours. As shown in FIG. 6A, the glucose-lowering effect of a single dose of ADISSP was maintained for more than 20 hours. The glucose-lowering effect of ADISSP protein was not due to any difference of food intake, as there was no food provided in the first 8 hours. 46 hours post the first injection, ob/ob mice were injected with ADISSP (50 nmole/kg) once every 48 hour at 8 am (arrow). Mice were fed ad libitum. Glucose was lowered by 83.5 mg/dL on average. As shown in FIG. 6B, after each subsequent injection, the glucose-lowering effect was maintained for at least 48 hours. Liver steatosis was also improved, as all of the livers in the treatment group were darker, indicating reduced lipid accumulation, and Liver histology showed reduced lipid accumulation (FIG. 6C). Finally, an improvement in hyperinsulinemia was also seen (FIG. 6D). These effects are not due to the presence of the His-tag on the ADISSP protein, as injection of conditioned medium containing ADISSP protein without the His-tag produced similar effects.

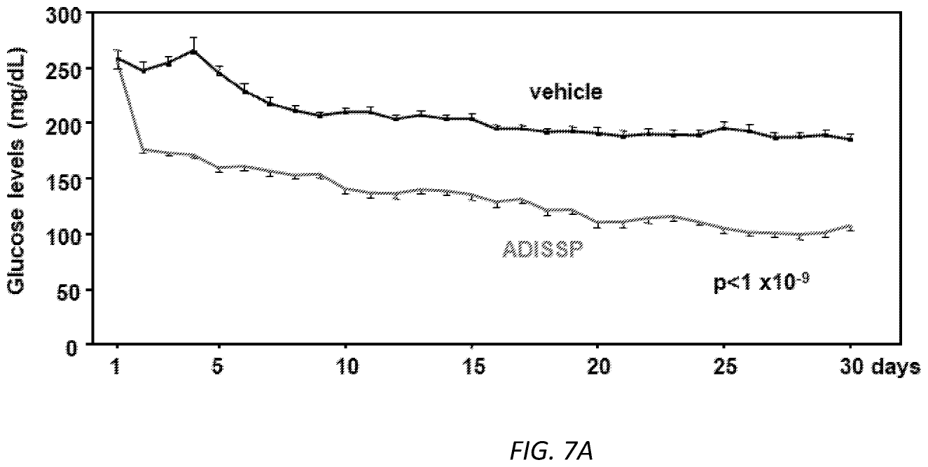

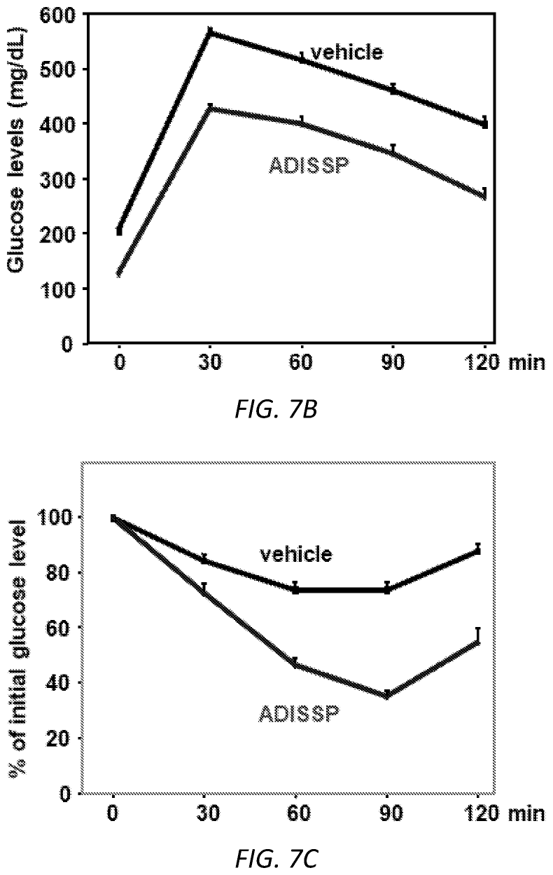

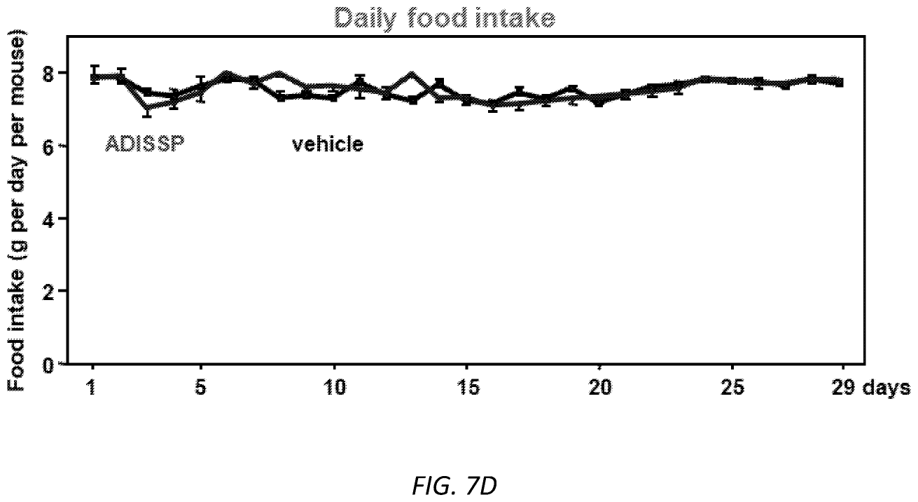

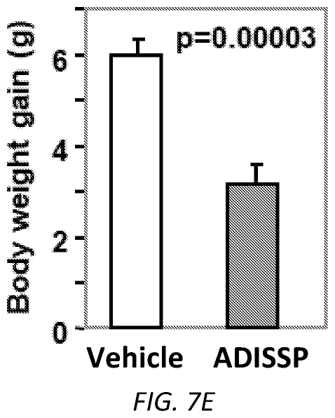

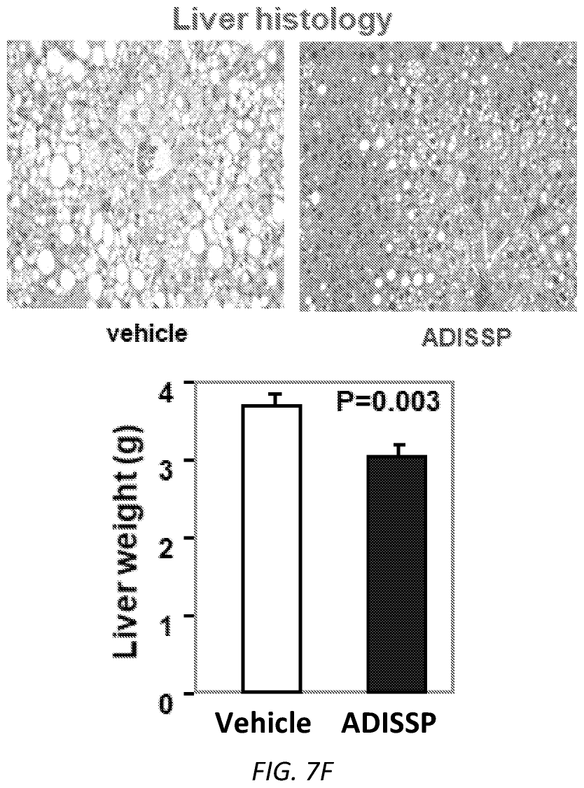

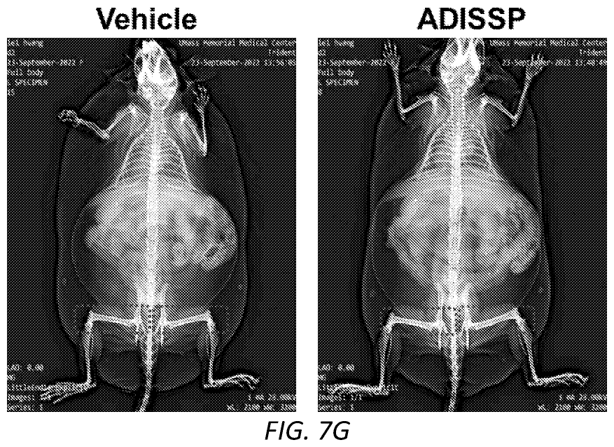

To evaluate longer-term effects of treatment with ADISSP, 12-week ob/ob mice (6 male and 8 female mice in control group and 8 male and 8 female mice in treatment group) were used. Glucose levels were measured at 7 am daily. Vehicle or ADISSP protein (62.6 nmole/kg per day) was ip injected once a day at 8 am for 30 days. The results, shown in FIGS. 7A-F, demonstrated that chronic treatment with ADISSP normalized hyperglycemia, improved glucose tolerance and insulin sensitivity, reduced body weight gain and ameliorated liver steatosis. Chronic treatment did not affect food intake. In addition, after one-month treatment of ob/ob mice with ADISSP protein, bone density was examined by X-Ray. We did not see decreased bone density. Instead, a slight increase was observed, as shown in the region in the box in FIG. 7G. Loss of bone density is associated with FGF21 and diabetes drug TZDs.

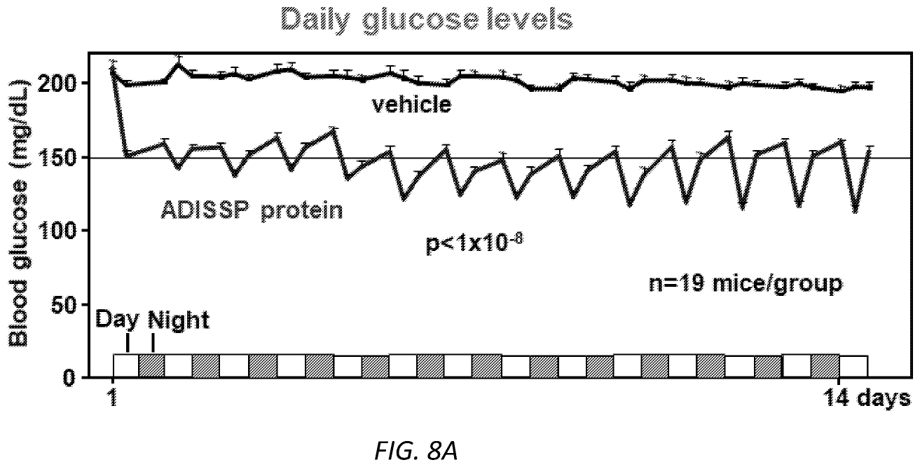

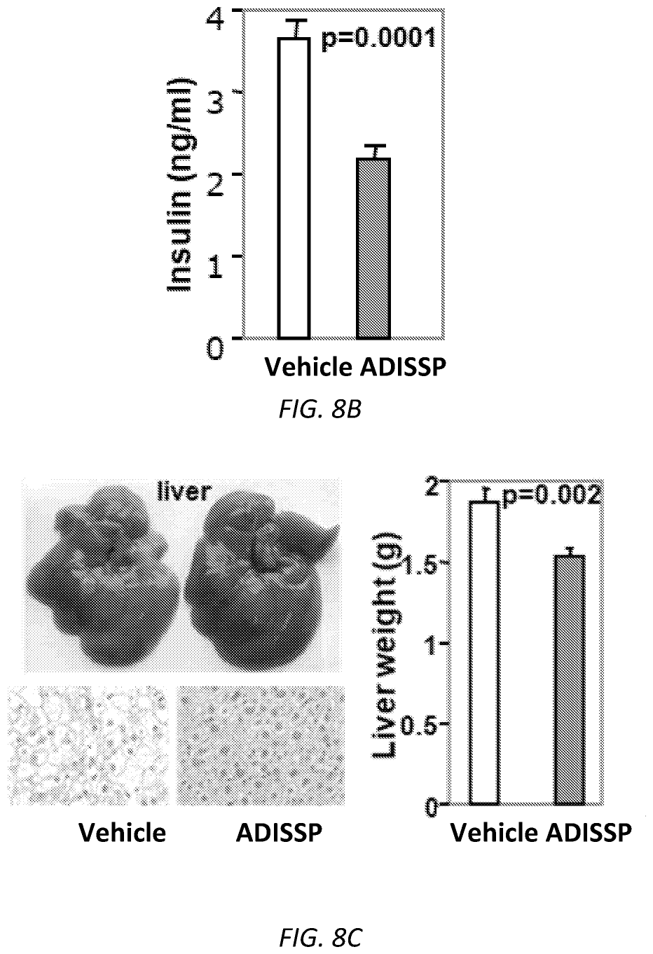

In the experiment shown in FIGS. 7A-G, all daily glucose levels were measured 24-hr post ADISSP injection. A much stronger glucose reduction would have been observed if measured at earlier time points, e.g., at 6-hr or 12-hr following protein injection. To demonstrate this and also investigate the benefits of ADISSP protein in a different type 2 diabetic mouse model, a two-week treatment course of once-daily administration with purified ADISSP protein was performed in high fat-induced obese (DIO) mice. Male mice (n=19 mice/group) were fed a high fat diet for 20 weeks to render them obese and hyperglycemia. Mice were then ip injected with ADISSP protein (65 nmole/kg) once a day at 8 am for a total of 14 injections. Glucose levels at ad libitum state were measured at 7:30 am, 2 μm and 8 μm. Insulin levels and livers were examined. This resulted in a clear improvement in daily glucose levels (FIG. 8A), more robustly at 2 μm (6 hr from injection) and 8 μm (12 hr from injection) time points than at 7:30 am (24 hr from injection), and reduced hyperinsulinemia (FIG. 8B), and amelioration of fatty liver (all of the livers were darker-less fatty-after ADISSP treatment) (FIG. 8C) and reduction of liver weight due to removal of fat from liver (FIG. 8C).

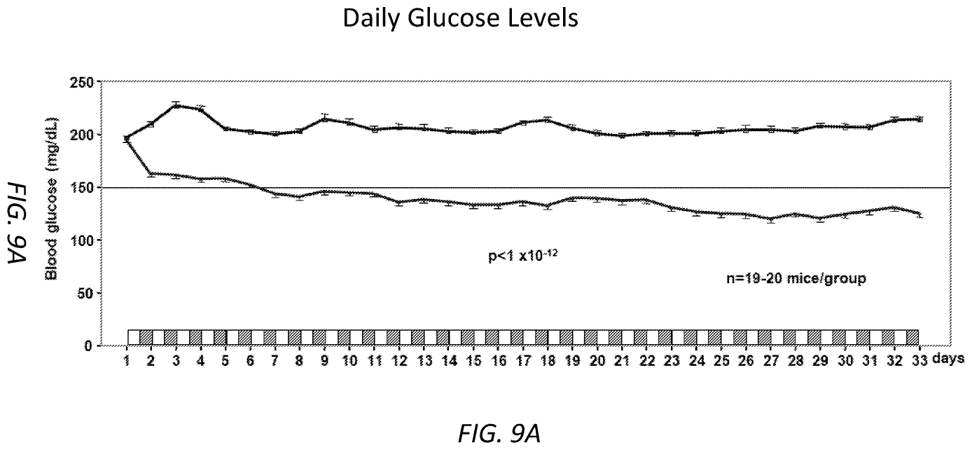

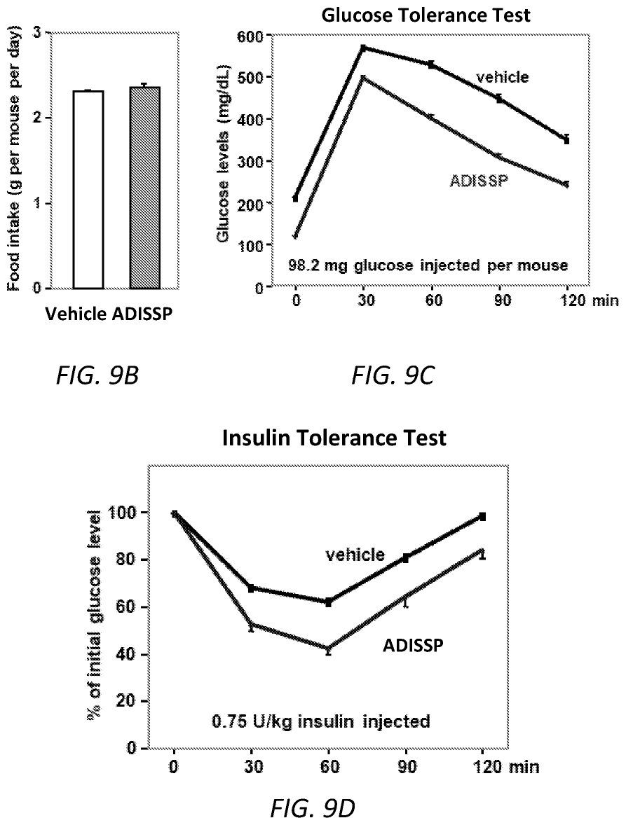

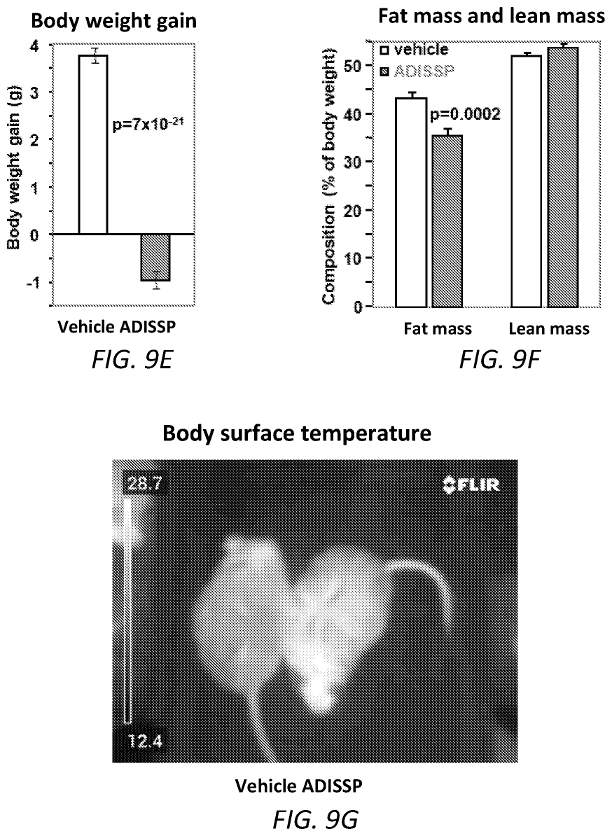

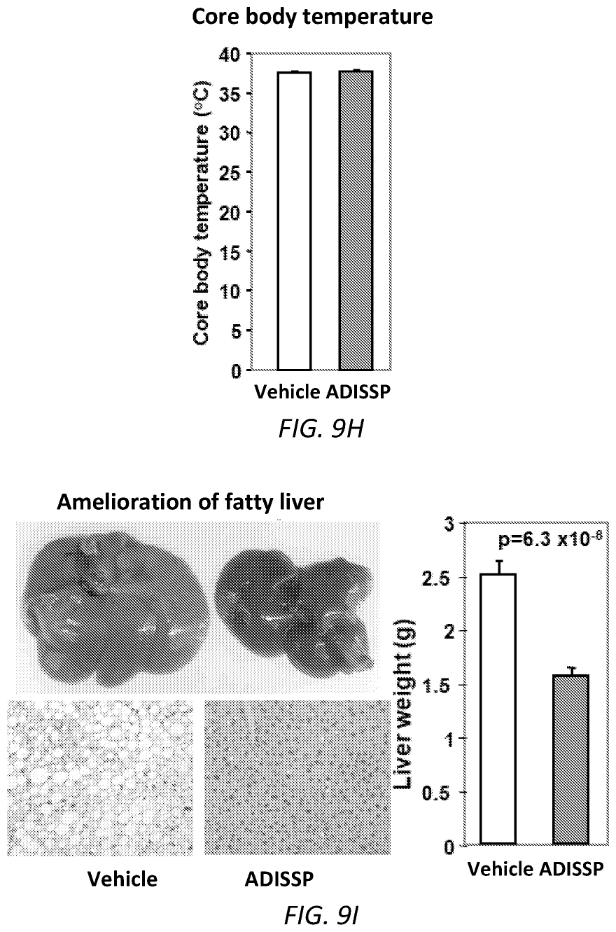

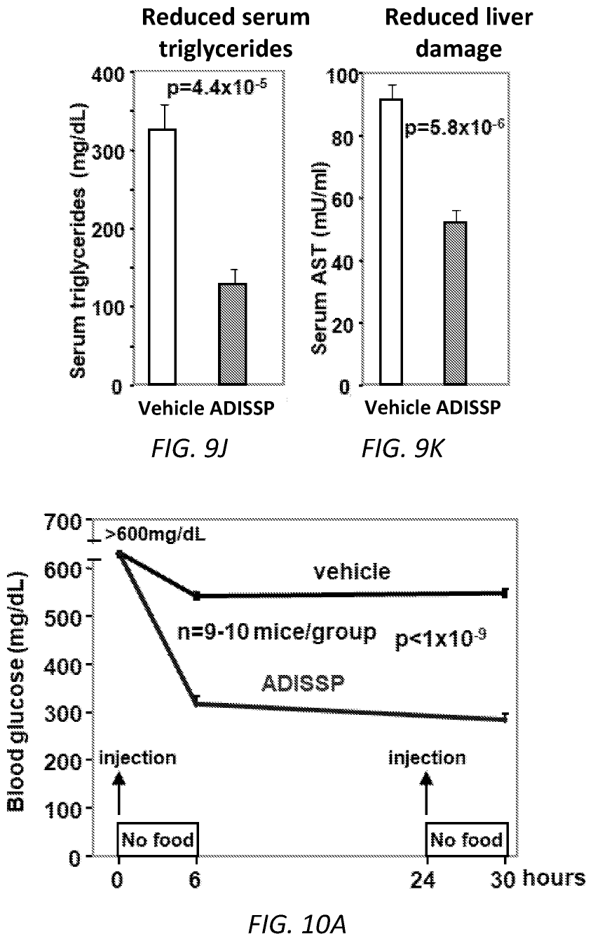

We next performed a one-month treatment of hyperglycemic DIO mice (n=19-20 DIO mice/group) with purified ADISSP protein. Glucose levels were measured every day at 7:30 am followed by daily intraperitoneal injection of ADISSP (71 nmole/kg per day). This treatment completely normalized hyperglycemia (FIG. 9A) but had no effect on food intake (FIG. 9B). Glucose tolerance (FIG. 9C) and insulin sensitivity (FIG. 9D) were markedly improved. While the vehicle-treated mice continued to gain weight, the ADISSP-treated mice displayed decreased body weight (FIG. 9E) along with a reduction of fat mass but not lean mass (FIG. 9F), which was due to increased thermogenesis in adipose tissue that dissipated energy as heat, as shown by higher surface body temperature (FIG. 9G) but normal core body temperature (FIG. 9H). ADISSP treatment completely reversed fatty liver and normalized liver weight (due to removal of fat from liver) (FIG. 9I). Hypertriglyceridemia (FIG. 9J) and high level of Aspartate aminotransferase (AST) (indicative of liver damage caused by fatty liver) (FIG. 9K) were normalized as well.

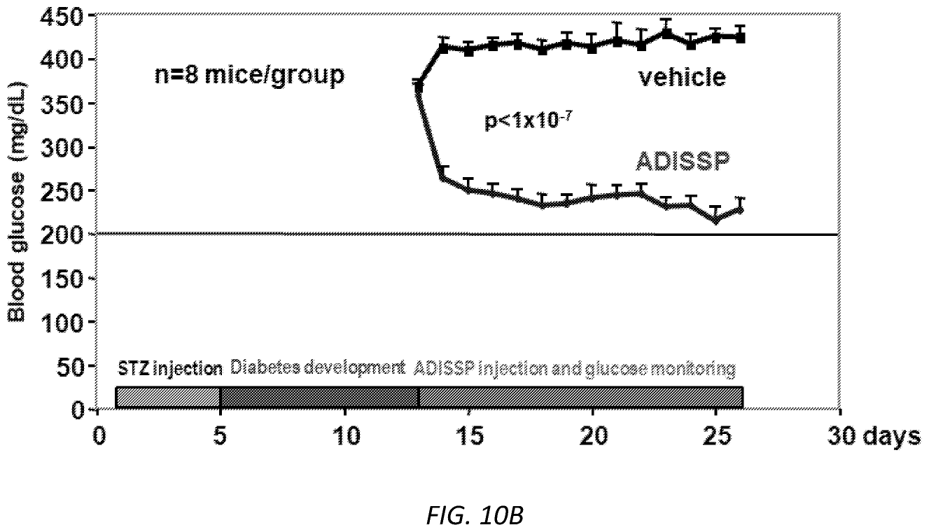

The effects of ADISSP protein administration on blood glucose and polyuria were evaluated in streptozotocin (STZ)-induced type 1 diabetic mice. A single dose (200 mg/kg) of STZ was ip injected into C57BL6 mice. Eight days post injection, as expected, high-dose STZ caused extremely severe type 1 diabetes with ad libitum glucose level (>600 mg/dL) exceeding the upper limit of the glucose meter. The mice were ip injected with ADISSP protein (50 nmole/kg) or vehicle at 0 hr and 24 h. After each injection, mice were fasted for 6 hours before glucose measurement so the glucose level in the vehicle group would fall below 600 mg/dL. The results showed 41-48% decrease of glucose levels (FIG. 10A) and improvement in polyuria. We were not able to continue the experiment because the mice were not healthy due to the high dose of STZ. To account for this, a low dose (50 mg/kg) of STZ was injected daily from Day 1 to Day 5 to produce healthy, type 1 diabetes mice. From Day 13, glucose was measured at 7:30 am followed by ip injection of ADISSP protein (70 nmole/kg) or vehicle once a day at 8 am for two weeks. At Day 13, glucose was measured after 2 hour fasting, all other time points were measured at ad libitum state. The results showed that ADISSP protein administration markedly lowered blood glucose (FIG. 10B) and improved polyuria in streptozotocin (STZ)-induced type 1 diabetic mice.

These results show that recombinant ADISSP protein, administered once a day at 1.0-1.4 mg/kg, effectively normalizes hyperglycemia, hyperinsulinemia, hypertriglyceridemia and fatty liver and reduces body weight in obese and diabetic mice. ADISSP protein also effectively lowers blood glucose in type 1 diabetic mice.

### Example 2. a Dissp Protein Treatment Resolves NASH and Cardiac Hypertrophy

While simple liver steatosis is a relatively benign condition, its more advanced stage, nonalcoholic steatohepatitis (NASH), initiated by lipid accumulation in hepatocytes and further characterized by lobular inflammation, hepatocyte ballooning, and varying degrees of fibrosis, can lead to cirrhosis, liver failure and cancer.

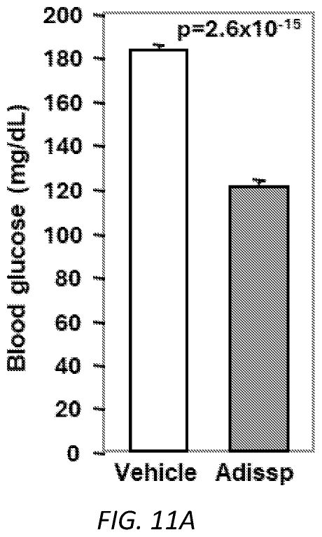

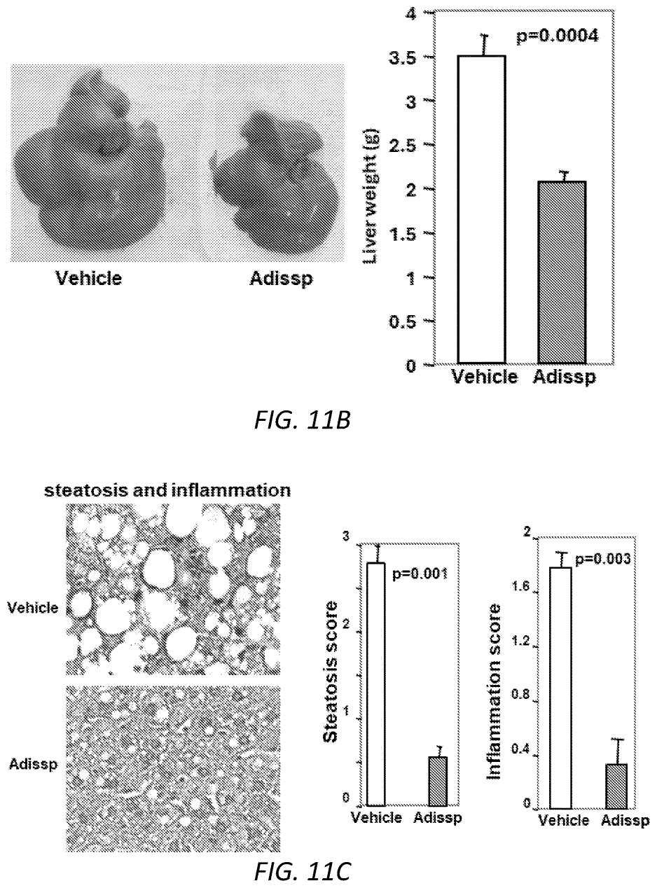

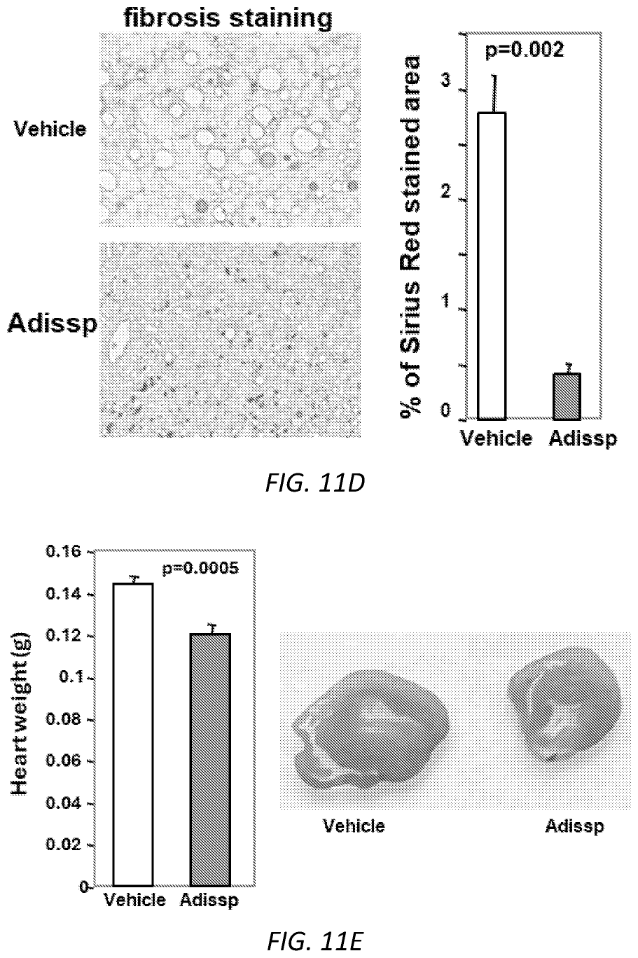

To examine the potential effects of Adissp protein on NASH, we fed wild-type male C57BL6J mice with the Gubra Amylin NASH (GAN) diet (Research Diets, D09100310) for 32 weeks to induce a NASH phenotype resembling human NASH. We then treated these mice with Adissp (113 nmole/kg per day) or vehicle. As expected, this treatment normalized hyperglycemia (FIG. 11A). At Day 16 of the treatment, a sub-group of mice were sacrificed and NASH phenotype was examined. We found that hepatomegaly (FIG. 11B), steatosis and inflammation were ameliorated (FIG. 11C). Moreover, fibrosis, as indicated by Sirius Red staining, was greatly alleviated (FIG. 11D). Thus, treatment with Adissp protein rapidly resolved the NASH phenotype.

The remaining mice were continuously treated with 63 nmole/kg per day Adissp protein or vehicle for 31 days. In addition to the benefits on liver NASH, Adissp protein also resolved high fat diet-induced cardiac hypertrophy (FIG. 11E), potentially improving cardiac function.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a mammalian subject who has insulin-dependent diabetes, Donohue syndrome, or Rabson-Mendenhall syndrome, the method comprising administering a therapeutically effective amount of adipose-secreted signaling protein (ADISSP) to a subject in need thereof.

2. The method of claim 1, wherein the subject has type 1 diabetes.

3. The method of claim 1, wherein the subject has a BMI of less than 25, at least 25, or at least 30.

4. The method of claim 1, wherein the subject is human.

5. The method of claim 4, comprising administering a polypeptide comprising a sequence that is at least 95% identical to SEQ ID NO:2.

6. The method of claim 5, wherein the polypeptide is administered once a day or once every other day.

7. The method of claim 6, wherein the polypeptide is administered parenterally.

8. The method of claim 7, wherein the polypeptide is administered intravenously, intramuscularly, or subcutaneously.

9. The method of claim 6, wherein the polypeptide is administered in a dose of 0.5-2.5 mg/kg/day, optionally 1.0-1.4 mg/kg per day.

10. The method of claim 1, wherein the polypeptide comprises one or more modifications.

11. The method of claim 10, wherein the modification comprises one or more of: replacement of one or more L amino acids with D amino acids; acetylation (optionally comprises an N-acetylalanine at position 2), amidation; conjugation to a linear or branched-chain monomethoxy poly-ethylene glycol (PEG); modification of the N- or C-terminus; glycosylation; polysialic acid (PSA) addition to a glycan; or fusion to a non-C20orf27 protein, optionally an Fc fusion proteins, fusion to human serum albumin, fusion to transferrin, or fusion to carboxy-terminal peptide of chorionic gonadotropin (CG) β-chain.

12. A method of treating a mammalian subject who has a disorder associated with obesity, preferably diabetes, metabolic syndrome, fatty liver disease, nonalcoholic steatohepatitis (NASH), non-alcoholic hepatic steatosis, obesity-associated cardiac hypertrophy, or dyslipidemia, the method comprising administering a therapeutically effective amount of adipose-secreted signaling protein (ADISSP) polypeptide to a subject in need thereof, wherein the polypeptide is administered in a dose of 0.5-2.5 mg/kg/day, optionally 1.0-1.4 mg/kg per day once a day or once every other day.

13. The method of claim 12, wherein the subject has a BMI of at least 25.

14. The method of claim 12, wherein the subject is human.

15. The method of claim 14, comprising administering a polypeptide comprising a sequence that is at least 95% identical to SEQ ID NO:2.

16. The method of claim 15, wherein the polypeptide is administered parenterally.

17. The method of claim 16, wherein the polypeptide is administered intravenously, intramuscularly, or subcutaneously.

18. The method of claim 12, wherein the polypeptide comprises one or more modifications.

19. The method of claim 18, wherein the modification comprises one or more of: replacement of one or more L amino acids with D amino acids; acetylation (optionally comprises an N-acetylalanine at position 2), amidation; conjugation to a linear or branched-chain monomethoxy poly-ethylene glycol (PEG); modification of the N- or C-terminus; glycosylation; polysialic acid (PSA) addition to a glycan; or fusion to a non-C20orf27 protein, optionally Fc fusion proteins, fusion to human serum albumin, fusion to transferrin, or fusion to carboxy-terminal peptide of chorionic gonadotropin (CG) β-chain.


SEQUENCE LISTING (2 sequences)

SEQ ID NO: 1 - protein, 199 aa, Homo sapiens
MAAANKGKCLPGVVGLAQALPVGPGRRAIAAGNKPRVRSIRFAAGHDAEGSHSHVHFDEKLHDSVVMVTQESDSSFLVKVGFLKILHRYEITFTLPPVHRLSKDVREAPVPSLHLKLLSVVPVPEGYSVKCEYSAHKEGVLKEEILLACEGGTGTCVRVTVQARVMDRHHGTPMLLDGVKCVGAELEYDSEHSDWHGFD

SEQ ID NO: 2 - protein, 174 aa, Homo sapiens
MAAANKGNKPRVRSIRFAAGHDAEGSHSHVHFDEKLHDSVVMVTQESDSSFLVKVGFLKILHRYEITFTLPPVHRLSKDVREAPVPSLHLKLLSVVPVPEGYSVKCEYSAHKEGVLKEEILLACEGGTGTCVRVTVQARVMDRHHGTPMLLDGVKCVGAELEYDSEHSDWHGFD